SEQUENCE LISTING (1 sequence)

SEQ ID NO: 1 - protein, 133 aa, Homo sapiens [sequence omitted]

(19) United States
(12) Patent Application Publication
(10) Pub. No.: US 2023/0277628 A1
(43) Pub. Date: Sep. 7, 2023

(54) METHODS FOR PROMOTING ANTI-TUMOR IMMUNE RESPONSE IN A SUBJECT IN NEED THEREOF USING ENCAPSULATED INTERLEUKIN 12

(71) Applicant: Spectrum Pharmaceuticals, Inc., Irvine, CA (US)

(72) Inventors: Sribalaji Lakshmikanthan, Boxborough, MA (US); Prasad Kolli, Mission Viejo, CA (US); Francois Lebel, Irvine, CA (US); John A. Barrett, Groton, MA (US)

(21) Appl. No.: 18/177,787

(22) Filed: Mar. 3, 2023

Related U.S. Application Data
(60) Provisional application No. 63/316,661, filed on Mar. 4, 2022.

(51) Int. Cl. A61K 38/20; A61P 35/00; A61P 37/04; A61P 25/00; A61K 9/50; A61N 5/10

(52) U.S. Cl. CPC A61K 38/208; A61K 9/5021; A61N 5/10; A61P 25/00; A61P 35/00; A61P 37/04

(57) ABSTRACT

The present invention relates to methods for promoting anti-tumor immune response in a subject in need thereof using encapsulated interleukin 12 (IL-12).

### CROSS-REFERENCE TO RELATED APPLICATIONS

This application claims priority under 35 U.S.C. §119(e) to U.S. Provisional Pat. Application No. 63/316,661, filed on Mar. 4, 2022. The foregoing application is incorporated by reference herein in its entirety.

### SEQUENCE LISTING

The sequence listing of the present application is submitted electronically as ST.26 formatted sequence listing with a file name “178923.00399.xml,” a creation date of Mar. 1, 2023, and a size of 1,892 bytes. This sequence listing submitted is part of the specification and is hereby incorporated by reference in its entirety.

### FIELD OF THE INVENTION

This invention relates to methods for promoting anti-tumor immune response in a subject in need thereof using encapsulated interleukin 12 (IL-12).

### BACKGROUND OF THE INVENTION

Failed T cell priming and high suppressor cell distributions in immunologically “cold” tumors commonly lead to resistance of immune checkpoint blockade (Vonderheide, 2018, Cancer Cell, 33(4):563-569; Jenkins et al., 2018, British Journal of Cancer, 118(1):9-16). The cytokine interleukin 12 (IL-12) is known for its anti-tumor potential by means of stimulating T cell activation immunogenic reprogramming of both lymphoid and myeloid-derived suppressor cells (Zeh et al., 1993, J Immunother Emphasis Tumor Immunol, 14(2):155-161; Suzuki et al., 1998, Tohoku J Exp Med, 185(3):223-226; Trinchieri et al., 1992, Prog Growth Factor Res, 4(4):355-368; Kerkar et al., 2011, J Clin Invest, 121(12):4746-4757). However, translational studies of IL-12 demonstrated underwhelming therapeutic effects, and trials were short-lived due to severe toxicities resulting from bolus systemic administration (Jenks, 1996, Journal of the National Cancer Institute, 88(9):576-577).

Accordingly, there exists a need for improved methods for prevention and treatment of cancer.

### SUMMARY OF THE INVENTION

This disclosure addresses the need mentioned above in a number of aspects. In one aspect, this disclosure provides a method of promoting anti-tumor immune response in a subject in need thereof. In some embodiments, the method comprises locally (e.g., intratumorally) administering to the subject a therapeutically effective amount of a pharmaceutical composition comprising encapsulated interleukin 12 (IL-12) in a local short term sustained release manner.

In some embodiments, the IL-12 is encapsulated in a semi-crystalline matrix. In some embodiments, the semi-crystalline matrix comprises at least one semi-crystalline water-soluble polymer in an amount of at least 30% by weight of the total mass of the semi-crystalline matrix. In some embodiments, the semi-crystalline water-soluble polymer is selected from polyalkylene glycols, polyalkylene glycol copolymers, polyvinyl alcohols, hydroxyalkyl celluloses, polysorbates, polyoxyethylene stearates poly, carrageenans and alginates, and a combination thereof.

In some embodiments, the pharmaceutical composition further comprises an excipient selected from the group consisting of poly(vinyl pyrrolidone) (PVP), surfactants, sucrose, and glycine. In some embodiments, the pharmaceutical composition further comprises a biocompatible polymer selected from polylactic acid, polyglycolic acid, poly(lactide-co-glycolide), poly(fumaric-co-sebacic anhydride), polycaprolactone, and blends or copolymers thereof. In some embodiments, the pharmaceutical composition further comprises an additional agent. In some embodiments, the pharmaceutical composition comprises at least one diluent or vehicle.

In some embodiments, the encapsulated IL-12 comprises a mouse IL-12. In some embodiments, the encapsulated IL-12 comprises a human IL-12.

In some embodiments, the method comprises local administration to the subject the encapsulated IL-12 in a single dosage of about 1 mg to about 2000 mg (equivalent to 1 ng to 2000 ng of recombinant human IL-12 (rhIL-12)). In some embodiments, the method comprises administering to the subject the encapsulated IL-12 in a single dosage of about 200 mg to about 1600 mg (equivalent to about 200 ng to about 1600 ng of rhIL-12).

In some embodiments, the anti-tumor immune response is against a condition selected from glioblastoma (GBM), Crohn’s disease, ulcerative colitis, irritable bowel syndrome, gastrointestinal cancer, and celiac disease.

In some embodiments, the method comprises administering the subject a second agent or therapy. In some embodiments, the second agent or therapy comprises the subject has been administered a radiation therapy prior to administration of the encapsulated IL-12. In some embodiments, the encapsulated IL-12 is administered to the subject before, after, or concomitantly with administration of the second agent or therapy.

In some embodiments, the radiation therapy comprises ionizing radiation. In some embodiments, the ionizing radiation is administered as a targeted radiation therapy (such as hypofractionated tumor directed radiotherapy and stereotactic body radiation therapy (SBRT)).

In some embodiments, the subject is a mammal. In some embodiments, the subject is a human. In some embodiments, the subject is locally immunocompromised. In some embodiments, the subject is immunoscenescent.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIGS. 1A, 1B, 1C, and 1D show tumor and serum concentrations of IL-12 and IFN-γ in the GL-261 orthotopic mouse model.

FIG. 2 shows that PCX12 treatment improves survival in the GL-261 orthotopic mouse model.

FIG. 3 shows therapeutic vaccine effect of PCX12 when rechallenged with GL-261 cells on the ipsilateral side of the brain.

### DETAILED DESCRIPTION OF THE INVENTION

This disclosure is based, at least in part, on unexpected discoveries that locally (e.g., intratumorally) administrated encapsulated IL-12 promotes anti-tumor immune response, thus reducing tumor growth and prolonging survival of a subject.

In one aspect, this disclosure provides a method of promoting anti-tumor immune response in a subject in need thereof. In some embodiments, the method comprises administering to the subject intratumorally a therapeutically effective amount of a pharmaceutical composition comprising encapsulated interleukin 12 (IL-12).

As used herein, the term “immune response” refers to the action of an immune cell, for example, lymphocytes, antigen-presenting cells, phagocytic cells, granulocytes, and soluble macromolecules (e.g., proteins, toxins, such as perforin, granzymes) produced by the above cells or the liver (including antibodies, cytokines, and complement), that results in selective damage to, destruction of, or elimination from the human body of invading pathogens, cells or tissues infected with pathogens, cancerous cells, or, in cases of autoimmunity or pathological inflammation, normal human cells or tissues. In some embodiments, an immune response comprises an antigen-specific T cell response.

The phrase “promoting, enhancing, or inducing an immune response” refers to causing or stimulating an immune cell (e.g., T cell, NK cells), dendritic cells and macrophages to have a sustained or amplified biological function. For example, induced or enhanced immune cell responses include increased production of cytokines by cytotoxic T cells (CD8⁺) or NK cells, increased proliferation, or increased antigen responsiveness relative to the response before intervention. In certain embodiments, the level of enhanced immune cell (e.g., T cell, NK cells) response after contact with a MNK-specific inhibitor is as least about 25%, 30%, 35%, 40%, 45%, 50%, 55%, 60%, 65%, 70%, 75%, 80%, 85%, 90%, 95%, 99%, or more, as compared to immune cells not contacted with the MNK-specific inhibitor. The assay for detecting cytokine levels (e.g., IL-2, IL-10, IFN-γ) to determine whether an immune response induced or enhanced is the multiplex assay described by Dossus et al. (J. Immunol. Methods 350:125, 2009). The assay for detecting T cell proliferation to determine whether an immune response induced or enhanced is the assay described by Liu et al. (Clin. Cancer Res. 21:1639, 2015). The assay for determining increased antigen responsiveness is the assay described by Tumeh et al. (Nature 515:568, 2014).

The term “promoting,” “enhancing,” or “inducing” in the context of an immune response refers to an increase in immune response, such as an increase in the ability of immune cells to target and/or kill cancer cells or to reduce or inhibit growth of cancer cells.

In eukaryotic cells, human IL2 is synthesized as a precursor polypeptide of 153 amino acids, from which 20 amino acids are removed to generate mature secreted IL2 (Taniguchi et al., 1983, Nature 302(5906):305-10). Mature human IL2 has the following amino acid sequence:

APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRW ITFCQSIISTLT(SEQ ID NO: 1)

In some embodiments, the IL2 as used in this disclosure can have amino acid deletions and/or substitutions selected from des-A1 M104A IL2, des-A1 M104A C125S IL2, M104A IL2, M104A C125A IL2, des-A1 M104A C125A IL2, and M104A C125S IL2, in addition to other variations alter the binding of IL2 to its receptor. These and other mutants may be found in U.S. Pat. No. 5,116,943 and in Weiger et al., 1989, Eur J Biochem 180:295-300.

In some embodiments, the IL2 may include an amino acid sequence having at least about 90%, at least about 91%, at least about 92%, about at least 93%, at least about 94%, at least about 95%, at least about 96%, at least about 97%, at least about 98%, at least about 99% or 100% sequence identity to mature human IL2, e.g., the human IL2 as set forth in SEQ ID NO: 1.

Examples of encapsulated IL-12 are described, e.g., in WO2020081929A1, WO2016025911A1, and U.S. Pat. Nos. 7,029,700; 8,524,829; 10,722,468, the disclosures of which are incorporated by reference in their entirety. In some embodiments, the IL-12 is encapsulated such as in cell encapsulation, vesicles encapsulation, or microfluidic encapsulation. In some embodiments, the IL-12 is encapsulated in a semi-crystalline matrix. In some embodiments, the semi-crystalline matrix comprises at least one semi-crystalline water-soluble polymer in an amount of at least 30% by weight of the total mass of the semi-crystalline matrix. In some embodiments, the semi-crystalline water-soluble polymer is selected from polyalkylene glycols, polyalkylene glycol copolymers, polyvinyl alcohols, hydroxyalkyl celluloses, polysorbates, polyoxyethylene stearates poly, carrageenans and alginates, and mixtures thereof.

In some embodiments, the pharmaceutical composition further comprises an excipient selected from the group consisting of poly(vinyl pyrrolidone) (PVP), surfactants, sucrose, and glycine. In some embodiments, the pharmaceutical composition further comprises a biocompatible polymer selected from polylactic acid, polyglycolic acid, poly(lactide-co-glycolide), poly(fumaric-co-sebacic anhydride), polycaprolactone, and blends or copolymers thereof. In some embodiments, the pharmaceutical composition further comprises an additional agent. In some embodiments, the pharmaceutical composition comprises at least one diluent or vehicle.

In some embodiments, the encapsulated IL-12 comprises rhIL-12; Oligo(D,L lactate-co-glycolate) 50:50 Mn 2300; Evonik Resomer Condensate RG 50:50 Mn 2300; Poly(D,L-lactide) IV 0.26-0.54 dL/g; Lactal Pluronic F127; Polyethylene glycol 3350; Polyethylene glycol 4500; Tween 20; Tween 80; Sucrose; Glycine; Polyvinylpyrrolidone K15; and Span 80.

In some embodiments, the encapsulated IL-12 comprises an IL-12 protein/polypeptide, an IL-12 variant, an IL-12 peptidomimetic, or a functional fragment of IL-12. In some embodiments, the encapsulated IL-12 comprises a mouse IL-12. In some embodiments, the encapsulated IL-12 comprises a human IL-12.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, a fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide; for example, unique subsequences (e.g., as determined by standard sequence comparison and alignment techniques) of the polynucleotides disclosed herein are also encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide; for example, unique subsequences (e.g., as determined by standard sequence comparison and alignment techniques) of the polypeptides disclosed herein are also encompassed by this disclosure.

A “functional variant” of a protein, as used herein, refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

The variants of IL-12 may be (i) one in which one or more of the amino acid residues are substituted with a conserved or non-conserved amino acid residue (e.g., a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The percent identity between two amino acid sequences can be determined using the algorithm of E. Meyers and W. Miller (Comput. Appl. Biosci., 4:11-17 (1988)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of this disclosure. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

For determination of protein sequence identify, the values included are those defined as ‘Identities″ by NCBI and do not account for residues that are not conserved but share similar properties. In some embodiments, the detectable tag can be an affinity tag. The term “affinity tag,” as used herein, relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3xFLAG tag, a SUMO tag, and MBP-tag (MBP: maltose-binding protein). Further examples of affinity tags can be found in Kimple et al., Curr Protoc Protein Sci. 2013 Sep 24; 73: Unit 9.9.

In some embodiments, the detectable tag can be conjugated or linked to the N- and/or C-terminus of IL-12. The detectable tag and the affinity tag may also be separated by one or more amino acids. In some embodiments, the detectable tag can be conjugated or linked to the variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate,” “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this disclosure include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins that are joined by a peptide bond or via several peptides. The fusion protein may also comprise a peptide linker between the two domains.

In some embodiments, the method comprises administering to the subject the encapsulated IL-12 in a single dosage of about 1 mg to 2000 mg (e.g., 10 mg, 20 mg, 30 mg, 40 mg, 50 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg) which is equivalent to about 1 ng to about 2000 ng of rhIL-12. In some embodiments, the method comprises administering to the subject the encapsulated IL-12 in a single dosage of about 200 mg to about 1600 mg.

In some embodiments, anti-tumor immune response is characterized by cytokine levels (e.g., IL-12, IL-10, IFNγ, IP-10). In some embodiments, anti-tumor immune response is characterized by a level of interferon (IFN)-γ.

In some embodiments, the subject is a mammal. In some embodiments, the subject is a human.

As used herein, “cancer,” “tumor,” and “malignancy” all relate equivalently to hyperplasia of a tissue or organ. If the tissue is a part of the lymphatic or immune system, malignant cells may include non-solid tumors of circulating cells. Malignancies of other tissues or organs may produce solid tumors. The methods described herein can be used in the treatment of lymphatic cells, circulating immune cells, and solid tumors.

Cancers that can be treated include tumors that are not vascularized or are not substantially vascularized, as well as vascularized tumors. Cancers may comprise non-solid tumors (such as hematologic tumors, e.g., leukemias and lymphomas) or may comprise solid tumors. The types of cancers to be treated with the disclosed compositions include, but are not limited to, carcinoma, blastoma and sarcoma, and certain leukemias or malignant lymphoid tumors, benign and malignant tumors, and malignancies, e.g., sarcomas, carcinomas, and melanomas. Also included are adult tumors/cancers and pediatric tumors/cancers.

Hematologic cancers are cancers of the blood or bone marrow. Examples of hematologic (or hematogenous) cancers include leukemias, including acute leukemias (such as acute lymphocytic leukemia, acute myelocytic leukemia, acute myelogenous leukemia, promyelocytic, myelomonocytic, monocytic, and erythroleukemia), chronic leukemias (such as chronic myelocytic (granulocytic) leukemia, chronic myelogenous leukemia, and chronic lymphocytic leukemia), polycythemia vera, lymphoma, Hodgkin’s disease, non-Hodgkin’s lymphoma (indolent and high-grade forms), myeloma Multiple, Waldenstrom’s macroglobulinemia, heavy chain disease, myelodysplastic syndrome, hairy cell leukemia, and myelodysplasia.

Solid tumors are abnormal masses of tissue that usually do not contain cysts or liquid areas. Solid tumors can be benign or malignant. The different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma and other sarcomas, synovium, mesothelioma, Ewing tumor, leiomyosarcoma, rhabdomyosarcoma, colon carcinoma, lymphoid malignancy, pancreatic cancer, breast cancer, lung cancer, ovarian cancer, prostate cancer, hepatocellular carcinoma, squamous cell carcinoma, basal cell carcinoma, adenocarcinoma, carcinoma of the sweat gland, medullary thyroid carcinoma, papillary thyroid carcinoma, sebaceous gland carcinoma of pheochromocytomas, carcinoma papillary, papillary adenocarcinomas, medullary carcinoma, bronchogenic carcinoma, renal cell carcinoma, hepatoma, bile duct carcinoma, choriocarcinoma, Wilms tumor, cervical cancer, testicular tumor, seminoma, bladder carcinoma, melanoma, and CNS tumors (such as glioma) (such as brainstem glioma and mixed gliomas), glioblastoma (also astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastasis).

Non-limiting examples of tumors can be treated by the methods described herein include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endothelio sarcoma, lymphangiosarcoma, lymphangioendotheliosarcoma, synovioma, testicular cancer, uterine cancer, cervical cancer, gastrointestinal cancer, mesothelioma, cancers associated with viral infection (such as but not limited to human papilloma virus (HPV) associated tumors (e.g., cancer cervix, vagina, vulva, head and neck, anal, and penile carcinomas)), Ewing’s tumor, leiomyosarcoma, Ewing’s sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom’s macroglobulinemia, papillary adenocarcinomas, cystadenocarcinoma, bronchogenic carcinoma, bile duct carcinoma, choriocarcinoma, seminoma, embryonal carcinoma, Wilms’ tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin’s lymphoma, Hodgkin’s lymphoma, Epstein-Barr virus (EBV) induced malignancies of all types including but not limited to EBV-associated Hodgkin’s and non-Hodgkin’s lymphoma, all forms of post-transplant lymphomas including post-transplant lymphoproliferative disorder (PTLD), uterine cancer, renal cell carcinoma, hepatoma, hepatoblastoma.

Cancers that may be treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lympho epithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget’s disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi’s sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing’s sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglio neuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin’s disease; Hodgkin’s lymphoma; paragranuloma; malignant lymphoma, small lymphocytic; malignant lymphoma, large cell, diffuse; malignant lymphoma, follicular; mycosis fungoides; other specified non-Hodgkin’s lymphomas; malignant histiocytosis; multiple myeloma; mast cell sarcoma; immunoproliferative small intestinal disease; leukemia; lymphoid leukemia; plasma cell leukemia; erythro leukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

### Combination Therapy

In some embodiments, the method comprises administering to the subject a second therapeutic agent or therapy, before, after, or concomittantly with administration of the encapsulated IL-12.

In some embodiments, the encapsulated IL-12 can be used in combination with another therapeutic treatment or agent to treat cancer. For example, the encapsulated IL-12 may be administered alone, or in combination with one or more therapeutically effective agents or treatments. The other therapeutically effective agent may be incorporated into the same composition as the encapsulated IL-12, or may be administered as a separate composition. The other therapeutically agent or treatment may be administered prior to, during and/or after the administration of the encapsulated IL-12.

In some embodiments, the encapsulated IL-12 is co-administered with one or more other therapeutic agents or treatments. In other embodiments, the encapsulated IL-12 is administered independently from the administration of one or more other therapeutic agents or treatments. For example, the encapsulated IL-12 is administered first, followed by the administration of one or more other therapeutic agents or treatments. Alternatively, one or more other therapeutic agents are administered first, followed by the administration of the encapsulated IL-12. As another example, a treatment (e.g., surgery etc.) is carried out first, followed by the administration of the encapsulated IL-12.

Other therapeutically effective agents/treatments include surgery, anti-neoplastics (including chemotherapeutic agents and radiation), anti-angiogenesis agents, antibodies to other targets, small molecules, photodynamic therapy, immunotherapy, immunity enhancing therapy, cytotoxic agents, cytokines, chemokines, growth inhibitory agents, anti-hormonal agents, kinase inhibitors, cardioprotectants, radiotherapeutics, immunostimulatory agents, immunosuppressive agents, and agents that promote proliferation of hematological cells.

In one embodiment, the “another therapeutic agent,” as used herein, are second, distinct therapeutic agents or anti-cancer agents, i.e., therapeutic agents or anti-cancer agents “other than” the encapsulated IL-12. Any additional therapeutic agent may be used in combination with the encapsulated IL-12. One or more additional therapeutic agents may be selected with a view to achieving additive, greater than additive and potentially synergistic effects, according to the following guidance.

To practice combined anti-tumor therapy, one would administer to an animal or patient the encapsulated IL-12 in combination with another distinct anti-cancer agent in a manner effective to result in their combined anti-tumor actions within the animal or patient. The agents would therefore be provided in amounts effective and for periods of time effective to result in their combined, or concurrent, presence within the tumor or tumor vasculature and their combined actions in the tumor environment. To achieve this goal, the encapsulated IL-12 and one or more additional, distinct anti-cancer agents may be administered to the animal substantially simultaneously, either in a single composition, or as two distinct compositions using different administration routes.

In one embodiment, administration of the encapsulated IL-12 may precede, or follow, concomitantly to administration of one or more additional anti-cancer agent by an interval ranging from seconds, to minutes, to hours, to days, to weeks.

The one or more additional therapeutic agents for separately timed combination therapies may be selected based upon certain criteria, including those discussed elsewhere herein. However, the selection of one or more distinct anti-cancer agents for prior or subsequent administration does not preclude their use in substantially simultaneous administration if desired.

Additional, distinct anti-cancer agents selected for administration “prior to” or “subsequent to” the encapsulated IL-12, and designed to achieve increased and potentially synergistic effects, include agents that benefit from the effects of the primary therapeutic agent. Accordingly, effective second, distinct anti-cancer agents for subsequent administration include anti-angiogenic agents, which inhibit metastasis; agents targeting necrotic tumor cells, such as antibodies specific for intracellular binding partner molecules that become accessible from malignant cells in vivo (U.S. Pat. Nos. 5,019,368, 4,861,581 and 5,882,626, each specifically incorporated herein by reference); chemotherapeutic agents; and anti-tumor cell immunoconjugates, which attack any tumor cells.

The encapsulated IL-12 can also be administered in combination with one or more additional cancer immunotherapy. The cancer immunotherapy can be one designed to elicit a humoral immune response against the subject’s cancer cells, or a cell-mediated immune response against the subject’s cancer cells, or a combination of a humoral response and a cell-mediated response against the subject’s cancer cells. Non-limiting examples of cancer immunotherapy useful in combination with the encapsulated IL-12 include a cancer vaccine, a DNA cancer vaccine, adoptive cellular therapy, adoptive immunotherapy, CAR T-cell therapy, antibodies, immunity enhancing compounds, cytokines, interleukins (e.g., IL-2, etc.), interferons (IFN-α, etc.), and checkpoint inhibitors (e.g., PD-1 inhibitor, PDL-1 inhibitor, CTLA-4 inhibitor, etc.).

In some situations, it may be desirable to extend the time period for treatment significantly, where several days (2, 3, 4, 5, 6 or 7), several weeks (1, 2, 3, 4, 5, 6, 7 or 8) or even several months (1, 2, 3, 4, 5, 6, 7 or 8) lapse between the respective administrations. This would be advantageous in circumstances where one treatment was intended to substantially destroy the tumor, and another treatment was intended to prevent metastasis or tumor re-growth. Anti-angiogenics should be administered at a careful time after surgery, however, to allow effective wound healing. Anti-angiogenic agents may then be administered for the lifetime of the patient.

Chemotherapeutic drugs can be used in combination with the encapsulated IL-12. Chemotherapeutic drugs can kill proliferating tumor cells, enhancing the necrotic areas created by the overall treatment.

A skilled artisan will understand that treating cancer in a patient includes, by way of non-limiting examples, killing and destroying a cancer cell, as well as reducing the proliferation of or cell division rate of a cancer cell. The skilled artisan will also understand that a cancer cell can be, by way of non-limiting examples, a primary cancer cell, a cancer stem cell, or a metastatic cancer cell. In some embodiments, one or more additional therapies for the cancer can be administered to the subject prior to, concurrently with, or subsequently to at least one of administration of the encapsulated IL-12. Examples of additional therapies that can be administered in addition to the encapsulated IL-12 include, but are not limited to, chemotherapeutic agents, antiproliferative agents, cytotoxic/anti-neoplastic agents, anti-angiogenic agents, and other anti-cancer agents.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene, and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacarbazine. Other cytotoxic/anti-neoplastic agents are antimetabolites for tumor cells, e.g., cytosine arabinoside, fluorouracil, methotrexate, mercaptopurine, azathioprine, and procarbazine. Other cytotoxic/anti-neoplastic agents are antibiotics, e.g., doxorubicin, bleomycin, dactinomycin, daunorubicin, mithramycin, mitomycin, mitomycin C, and daunomycin. There are numerous liposomal formulations commercially available for these compounds. Still other cytotoxic/antineoplastic agents are mitotic inhibitors (vinca alkaloids). These include vincristine, vinblastine, and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers, and antisense oligonucleotides. Other known inhibitors of angiogenesis include angiostatin, endostatin, interferons, interleukin 1 (including alpha and beta), retinoic acid, and tissue inhibitors of metalloproteinase-1 and -2 (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; albumin-bound paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; j asplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyna; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromely sin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; imilimumab; mirtazapine; BrUOG 278; BrUOG 292; RAD0001; CT-011; folfirinox; tipifarnib; R115777; LDE225; calcitriol; AZD6244; AMG 655; AMG 479; BKM120; mFOLFOX6; NC-6004; cetuximab; IM-C225; LGX818; MEK162; BBI608; MEDI4736; vemurafenib; ipilimumab; ivolumab; nivolumab; panobinostat; leflunomide; CEP-32496; alemtuzumab; bevacizumab; ofatumumab; panitumumab; pembrolizumab; rituximab; trastuzumab; STAT3 inhibitors (e.g., STA-21, LLL-3, LLL12, XZH-5, S31-201, SF-1066, SF-1087, STX-0119, cryptotanshinone, curcumin, diferuloylmethane, FLLL11, FLLL12, FLLL32, FLLL62, C3, C30, C188, C188-9, LYS, OPB-31121, pyrimethamine, OPB-51602, AZD9150, etc.); hypoxia inducing factor 1 (HIF-1) inhibitors (e.g., LW6, digoxin, laurenditerpenol, PX-478, RX-0047, vitexin, KC7F2, YC-1, etc.) and zinostatin stimalamer.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, a “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental animal or animal suitable as a disease model.

The terms “treat,” “treating,” and “treatment,” refer to therapeutic or preventative measures described herein. The methods of “treatment” employ administration to a subject, in need of such treatment, for example, a subject afflicted a disease or disorder, including pancreatic cancer, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

As used herein, “preventing” or “prevention” refers to any methodology where the disease state does not occur due to the actions of the methodology (such as, for example, administration of a probiotic and/or a prebiotic as described herein). In one aspect, it is understood that prevention can also mean that the disease is not established to the extent that it occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g., a subject who does not receive a probiotic and/or a prebiotic as described herein).

An “effective amount” or “therapeutically effective amount” refers to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

The term “disease” as used herein is intended to be generally synonymous, and is used interchangeably with the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “decrease,” “reduced,” “reduction,” “decrease,” or “inhibit” are all used herein generally to mean a decrease by a statistically significant amount. However, for avoidance of doubt, “reduced”, “reduction,” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “modulate” is meant to refer to any change in biological state, i.e., increasing, decreasing, and the like.

The terms “increased,” “increase,” “enhance,” or “activate” are all used herein to generally mean an increase by a statically significant amount; for the avoidance of any doubt, the terms “increased,” “increase,” “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein, generally refer to a biological material being tested for and/or suspected of containing an analyte of interest such as antibodies. The sample may be any tissue sample from the subject. The sample may comprise protein from the subject.

The terms “inhibit” and “antagonize,” as used herein, mean to reduce a molecule, a reaction, an interaction, a gene, an mRNA, and/or a protein’s expression, stability, function or activity by a measurable amount or to prevent entirely. Inhibitors are compounds that, e.g., bind to, partially or totally block stimulation, decrease, prevent, delay activation, inactivate, desensitize, or down-regulate a protein, a gene, and an mRNA stability, expression, function and activity, e.g., antagonists.

“Parenteral” administration of a composition includes, e.g., subcutaneous (s.c.), intravenous (i.v.), intramuscular (i.m.), or intrasternal injection, or infusion techniques.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism.

Multiple techniques of administering a compound exist in the art including, but not limited to, intravenous, intratumoral, oral, aerosol, parenteral, ophthalmic, pulmonary and topical administration.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### Intratumorally Administrated Encapsulated IL-12 (PCX12) Promotes Anti-Tumor Immune Response in GL-261 Murine Orthotopic Glioma Model

Glioblastoma (GBM) is inherently immunosuppressed (cold). To overcome this challenge, short-term localized controlled immune activation should turn the tumor hot by increasing cytotoxic T cell infiltrate, resulting in reduced tumor growth and prolonged survival (Barrett, J. A., et al. Cancer Gene Ther 25(5-6): 106-116 (2018)). An encapsulated-mouse IL-12 formulation (mPCX12) has been developed, using a sustained IL-12 release platform (Egilmez, N. K., et al. Cancer Res 60(14): 3832-3837 (2000)) to facilitate short-term localized immune activation in the tumor without systemic toxicity. mPCX12 elicited a sustained dose-related IL-12 release concomitant with downstream IFNγ in mouse splenocytes demonstrating mPCX12 biologic activity. Similar results were observed with hPCX12 in human PBMCs.

In an orthotopic murine glioma model, C57B6 mice were inoculated with 3 × 10⁵ GL-261 glioma cells in the frontal lobe via intracranial injection with drug treatment on Day 5. Mice were randomly assigned (n=22/group) to receive a single intratumoral dose of mPCX12 at 0.5, 0.75, and 1 mg, or mrIL-12 1 µg (equivalent to mPCX12 1 mg) or empty shells or vehicle. In addition, lomustine (6 mg/kg, QDx5 i.p.) was also assessed. The results showed that mPCX12 treatment increased tumor IL-12 and downstream IFNγ levels in a dose-dependent manner. At 1 mg mPCX12, tumor IL-12 and IFNy levels were 2069±631 and 646±8 pg/mg respectively on Day 3. Low levels of IL-12 and IFNy observed in the systemic circulation of mPCX12 and mrIL-12 groups. mPCX12 tumor IL-12 persisted while mrIL-12 returned to baseline by Day 7. Flow cytometric immunophenotyping of tumors showed increased cytotoxic T cells in mPCX12 treated gliomas. These increase in local cytokine levels translated into a dose-related prolongation of survival when compared to the median survival in the vehicle and empty shell groups (~19 days) and 20 days for lomustine, and 37 days in mrIL-12. mPCX12 elicited a dose-related increase in survival. mPCX12 at 1 mg dose, the median survival was > 90 days. At Day 90, 41%, 45%, and 55% of the animals that received doses of 0.5, 0.75 or 1 mg mPCX12, respectively, were alive with median survival >90 days (last day of study).

At the end of the study, subgroups were euthanized, and those animals treated with mPCX12 were tumor-free. The remaining surviving mPCX12 animals, 0.75 mg, and 1 mg dose groups, were reinoculated with 3 × 10⁵ GL261 glioma cells in the ipsilateral side of the brain and compared with age-matched control groups consisting of vehicle or empty shells. The median survival in control groups was 27 days, and rechallenged mPCX12 treated groups survived 100% at Day 90, demonstrating memory T cell activation.

In summary, short-term controlled release of locally administered IL-12 via PCX12 resulted in turning cold tumors hot, resulting in enhanced overall survival in the orthotopic GBM model.

In the first study, six days post GL-261 inoculation, a single dose of mPCX12 at 0.5, 0.75, and 1 mg was administered to 4 animals per timepoint (TP). Control treatment groups were, mrIL-12 1 µg (equivalent to mPCX12 1 mg) or empty shells. In addition, lomustine (6 mg/kg, QDx5 i.p.) was also assessed. At 3- and 7-days post drug administration, animals were euthanized, serum and tumor IL-12 and IFN-γ levels were determined via ELISA.

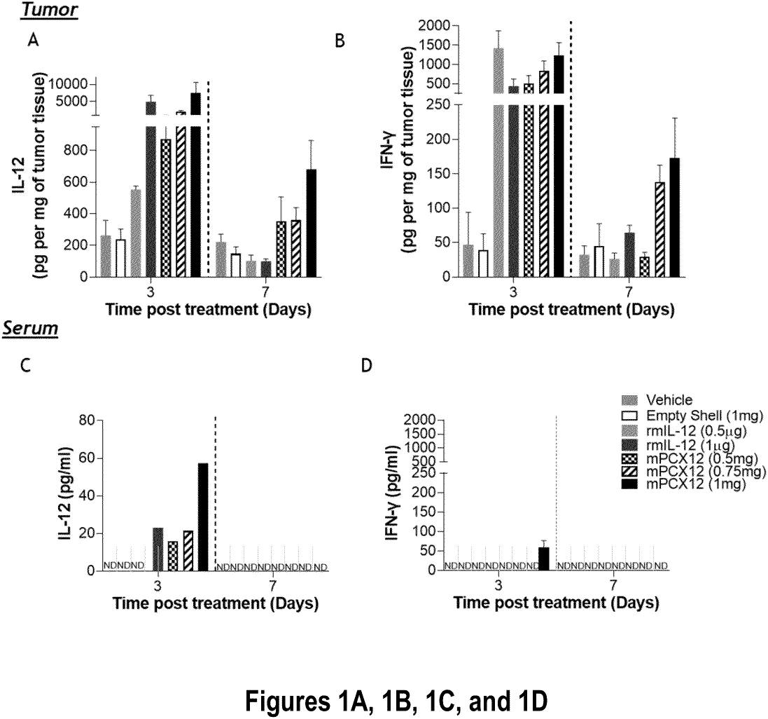

FIGS. 1 shows tumor and serum concentrations of IL-12 and IFN-γ in the GL-261 orthotopic mouse model. As desmonstrated, mPCX12 treatment increased tumor IL-12 and downstream IFN-γ levels demonstrating biologic activity in a dose dependent manner at Day 3 and Day 7. mPCX12 tumor IL-12 and IFN-γ persisted while mrIL-12 returned to baseline by Day 7. Low levels of IL-12 and IFN-γ observed in the systemic circulation of mPCX12 and mrIL-12 groups. FIGS. 1A-D: All values expressed as Mean +/- SEM; n=4 per timepoint; ND: Not Detected.

In a second study, GL-261 inoculated mice (n=22 per group) received a single dose of mPCX12 at 0.5, 0.75, and 1 mg. Control treatment groups were, mrIL-12, 0.5 and 1 µg (equivalent to mPCX12 0.5 and 1 mg) or empty shells (1 mg) or vehicle control intratumoral. In addition, lomustine (6 mg/kg, QDx5 i.p.) was also assessed. Day 71 was the last day of the study. Animals were euthanized when they became moribund.

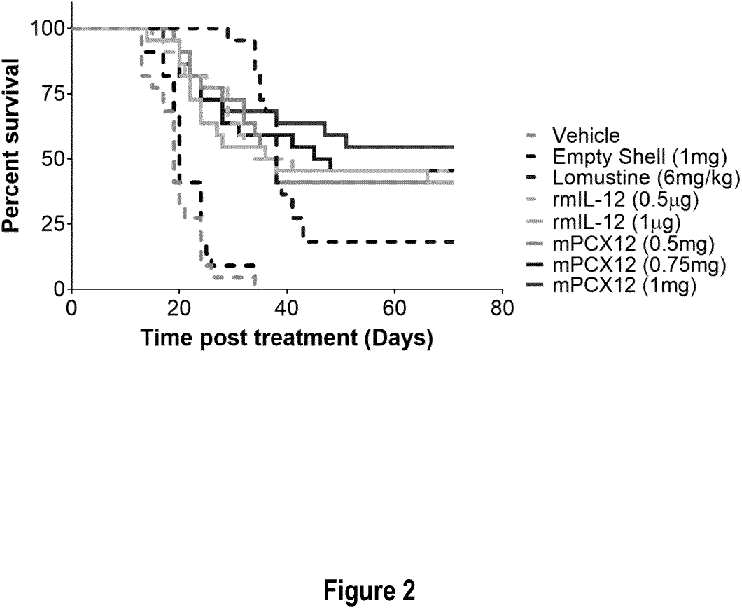

FIG. 2 and Table 1 show that PCX12 treatment improves survival in the GL-261 orthotopic mouse model. As demonstrated, PCX12 treated groups demonstrated dose dependent increase in over all survival. PCX12 treatment showed superiority to the current standard-of-care and rmIL-12. PCX12 (1 mg) >50% of mice survived to the end of the study (Day 71) and were tumor-free. PCX12 treated groups showed no change in clinical signs and body weight.

In a third study, on Day 93, surviving PCX12 administered mice from the main study (0.75 mg group: n=4 and 1 mg dose group: n=8) were inoculated with GL-261 cells (3×105 cells/mouse) on the ipsilateral side of the frontal lobe with intracranial injection. Age-matched control groups (n=8 per group) were implanted GL-261 cells 3×105 cells/mouse). Empty Shell group received a single dose of 1 mg equivalent of empty shells. The experiment continued for additional 100 Days.

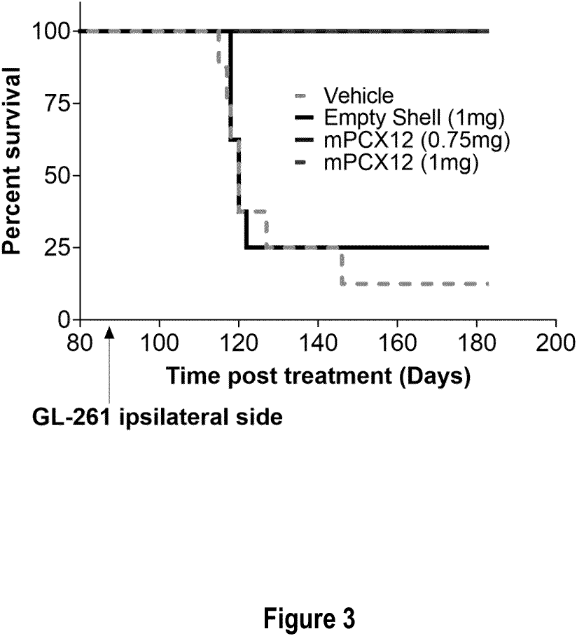

FIG. 3 and Table 2 show that PCX12 demonstrated a therapeutic vaccine effect when rechallenged with GL-261 cells on the ipsilateral side of the brain. It was found that PCX12 elicited similar time-dependent release kinetics in both human and murine encapsulated spheres. mPCX12 treatment increased tumor IL-12 and downstream IFN-γ levels demonstrating biologic activity in a dose dependent manner. mPCX12 treated groups demonstrated dose dependent increase in overall survival and was superior to current standard-of-care or mrIL-12. In the high dose PCX12 group >50% of mice survived to the end of study and were tumor-free. Surviving PCX12 treated mice when rechallenged with GL-261 cells in the ipsilateral side of the brain all survived and remained tumor-free for additional 100 Days (end of study).

It was concluded that short-term controlled release of locally administered PCX12 reactivated tumor microenvironment resulting in increased overall survival in the orthotopic GL-261 mouse model. When rechallenged with GL-261 cells in the ipsilateral side of the brain gliomas, PCX12 previously treated mice all survived until the end of study (100 days) and remained tumor-free, demonstrating therapeutic vaccine effect. PCX12 warrants further investigation in GBM patients.

TABLE 1

Median Survival and number of survivors at the end of the study Day 71

| Column1 | Control | Empty Shell (1 mg) | Lomustine (6 mg/kg; QDx5 .i.p.) | rmIL-12 (0.5 µg) | rmIL-12 (1 µg) | mPCX12 (0.5 mg) | mPCX12 (0.75 mg) | mPCX12 (1 mg) |
|---|---|---|---|---|---|---|---|---|
| n | 22 | 22 | 22 | 22 | 22 | 22 | 22 | 22 |
| Median survival (Days) | 19 | 20 | 38 | 38 | 37 | 37 | 47 | >71* |
| # censored mice | 0 | 0 | 4 | 10 | 9 | 9 | 10 | 12 |
| % alive at 71 Days Post Treatment | 0 | 0 | 18 | 45 | 41 | 41 | 45 | 55 |

*>50% survival at the end of the study

TABLE 2

Median Survival and number of survivors at the end of the study

| Rechallenge Study | Control | Empty Shell (1 mg) | mPCX12 (0.75 mg) | mPCX12 (1 mg) |
|---|---|---|---|---|
| n | 8 | 8 | 4 | 8 |
| Median survival (Days) | 27 | 27 | >100* | >100* |
| # censored mice | 1 | 2 | 4 | 8 |
| % alive at 69 days Post treatment | 13 | 25 | 100 | 100 |

* 100% survival at end of study. Mice were tumor free

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of promoting anti-tumor immune response in a subject in need thereof, comprising locally administering to the subject a therapeutically effective amount of a pharmaceutical composition comprising encapsulated interleukin 12 (IL-12).

2. The method of claim 1, wherein the encapsulated IL-12 is administered to the subject intratumorally.

3. The method of claim 1, wherein the IL-12 is encapsulated in a semi-crystalline matrix.

4. The method of claim 1, wherein the semi-crystalline matrix comprises at least one semi-crystalline water-soluble polymer in an amount of at least 30% by weight of the total mass of the semi-crystalline matrix.

5. The method of claim 3, wherein the semi-crystalline water-soluble polymer is selected from polyalkylene glycols, polyalkylene glycol copolymers, polyvinyl alcohols, hydroxyalkyl celluloses, polysorbates, polyoxyethylene stearates poly, carrageenans and alginates, and a combination thereof.

6. The method of claim 1, wherein the pharmaceutical composition further comprises an excipient selected from the group consisting of poly(vinyl pyrrolidone) (PVP), surfactants, sucrose, and glycine.

7. The method of claim 1, wherein the pharmaceutical composition further comprises a biocompatible polymer selected from polylactic acid, polyglycolic acid, poly(lactide-co-glycolide), poly(fumaric-co-sebacic anhydride), polycaprolactone, and blends or copolymers thereof.

8. The method of claim 1, wherein the pharmaceutical composition further comprises an additional agent.

9. The method of claim 1, wherein the pharmaceutical composition comprises at least one diluent or vehicle.

10. The method of claim 1, wherein the encapsulated IL-12 comprises a mouse IL-12.

11. The method of claim 1, wherein the encapsulated IL-12 comprises a human IL-12.

12. The method of claim 1, comprising administering to the subject the encapsulated IL-12 in a single dosage of about 1 mg to about 2000 mg.

13. The method of claim 1, comprising administering to the subject the encapsulated IL-12 in a single dosage of about 200 mg to about 1600 mg.

14. The method of claim 1, wherein anti-tumor immune response is against a condition selected from glioblastoma (GBM), Crohn’s disease, ulcerative colitis, irritable bowel syndrome, gastrointestinal cancer, and celiac disease.

15. The method of claim 1, further comprising administering the subject a second agent or therapy.

16. The method of claim 1, wherein the second agent or therapy comprises a radiation therapy.

17. The method of claim 15, wherein the encapsulated IL-12 is administered to the subject before, after, or concomitantly with administration of the second agent or therapy.

18. The method of claim 16, wherein the radiation therapy comprises ionizing radiation.

19. The method of claim 18, wherein the ionizing radiation is administered as a targeted radiation therapy.

20. The method of claim 1, wherein the subject is a human.
